(19) United States
(12) Patent Application Publication
(10) Pub. No.: US 2010/0203587 A1
(43) Pub. Date: Aug. 12, 2010

(54) USE OF DNA GYRASE INHIBITORS FOR IN VITRO POLYPEPTIDE SYNTHESIS REACTIONS

(75) Inventors: Alexei M. Voloshin, Newark, CA (US); James F. Zawada, Redwood City, CA (US); Daniel Gold, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Sutro Biopharma, Inc., South San Francisco, CA (US)

(21) Appl. No.: 12/685,989

(22) Filed: Jan. 12, 2010

Related U.S. Application Data
(60) Provisional application No. 61/144,401, filed on Jan. 13, 2009.

(51) Int. Cl. C12P 21/00; C12N 15/74

(52) U.S. Cl. 435/69.1; 435/320.1

(57) ABSTRACT

The present invention provides methods and compositions useful for in vitro polypeptide synthesis reactions. The methods involve the use of DNA gyrase inhibitors to prevent bacterial contamination in lysates used for in vitro production of polypeptides. The compositions include contamination-free cell lysates for in vitro protein synthesis reactions.

### RELATED APPLICATIONS

This application claims priority to U.S. Provisional Patent Application No. 61/144,401, filed Jan. 13, 2009, the contents of which are incorporated by reference in the entirety for all purposes.

### FIELD OF THE INVENTION

This invention relates to in vitro polypeptide synthesis. In particular, the invention relates to methods of using antibiotics that are DNA gyrase inhibitors during the in vitro polypeptide synthesis. The invention also provides compositions that are lysates containing DNA gyrase inhibitors for in vitro polypeptide synthesis reactions. The use of DNA gyrase inhibitors during in vitro polypeptide synthesis reactions enhances output of the desired protein. The present invention is particularly useful in an in vitro or cell-free system where protein synthesis is directed by the T7 bacteriophage promoter.

### BACKGROUND OF THE INVENTION

Protein synthesis is a fundamental biological process that underlies the development of polypeptide therapeutics, vaccines, diagnostics, and industrial enzymes. With the advent of recombinant DNA (rDNA) technology, it has become possible to harness the catalytic machinery of the cell to produce a desired protein. This can be achieved within the cellular environment or in vitro using lysates derived from cells.

In vitro, or cell-free, protein synthesis offers several advantages over conventional in vivo protein expression methods. Cell-free systems can direct most, if not all, of the metabolic resources of the cell towards the exclusive production of one protein. Moreover, the lack of a cell wall and membrane components in vitro is advantageous because it allows for control of the synthesis environment. For example, tRNA levels can be changed to reflect the codon usage of genes being expressed. The redox potential, pH, or ionic strength can also be altered with greater flexibility than with in vivo protein synthesis because concerns of cell growth or viability do not exist. Furthermore, direct recovery of purified, properly folded protein products can be easily achieved.

The productivity of cell-free systems has improved over two orders of magnitude in recent years, from about 5 μg/ml-hr to about 150, 250, or 500 μg/ml-hr. Such improvement has made in vitro protein synthesis a practical technique for laboratory-scale research and provided a platform technology for high-throughput protein expression. It further indicates the feasibility for using cell-free technologies as an alternative means to in vivo large-scale commercial production of protein pharmaceuticals.

The productivity of in vitro polypeptide synthesis systems can be significantly hindered by bacterial contamination. While many commercially available antibiotics exist for use to inhibit bacterial growth that occurs within an in vitro reaction lysate, most of these antibiotics interfere with protein synthesis or are not acceptable for production of pharmaceuticals. There exists a need for antibiotic application for eliminating bacterial growth in an in vitro polypeptide synthesis reaction lysate without interfering with the in vitro protein synthesis reaction, such that the output of the desired polypeptide is permitted and/or enhanced. The invention described herein fulfills these needs, as will be apparent upon review of the following disclosure.

### BRIEF SUMMARY OF THE INVENTION

In one aspect, this invention provides a method for in vitro synthesis of a polypeptide. The method includes the step of adding at least one DNA gyrase inhibitor to a polypeptide synthesis reaction lysate in an amount sufficient to inhibit bacterial growth, especially in a reaction lysate where the polypeptide is synthesized from an expression cassette comprising a polynucleotide sequence encoding the polypeptide, where the coding sequence is operably linked to a T7 promoter. The DNA gyrase inhibitor may be a quinolone or an aminocoumarin.

In some embodiments, ciprofloxacin, one of the quinolones, is used in the methods of the present invention. In other embodiments, another quinolone, norfloxacin, is used. Other quinolones useful in the methods of this invention include cinoxacin, flumequine, nalidixic acid, oxolinic acid, piromidic acid, pipemidic acid, rosoxacin, enoxacin, fleroxacin, iomefloxacin, nadifloxacin, ofloxacin, pefloxacin, rufloxacin, balofloxacin, gatifloxacin, grepafloxacin, levofloxacin, moxifloxacin, pazufloxacin, sparfloxacin, temafloxacin, tosufloxacin, clinafloxacin, garenoxacin, gemifloxacin, sitafloxacin, trovafloxacin, prolifloxacin, and ecinofloxacin.

In some embodiments, coumermycin A₁, one of the aminocoumarins, is used in the methods of the present invention. In other embodiments, another aminocoumarin, novobiocin, is used. Clorobiocin is an additional aminocoumarin that may be useful in the cell-free protein synthesis method of this invention. Any combination of two or more of quinolones and/or aminocoumarins, such as those named above, can be used in the method of this invention.

The method of the present invention may be used to suppress the growth of either facilitative anaerobic microorganisms or aerobic microorganisms.

In a second aspect, the present invention provides an in vitro polypeptide synthesis reaction lysate or mixture, which contains at least one DNA gyrase inhibitor in an amount sufficient to inhibit bacterial growth. In particular, this reaction mixture contains an expression cassette that includes a polynucleotide sequence encoding a polypeptide to be synthesized, operably linked to a T7 promoter. The DNA gyrase inhibitor may be a quinolone or an aminocoumarin.

In some embodiments, ciprofloxacin, one of the quinolones, is used in the reaction mixtures of the present invention. In other embodiments, another quinolone, norfloxacin, is used. Other quinolones useful in the reaction mixtures of this invention include cinoxacin, flumequine, nalidixic acid, oxolinic acid, piromidic acid, pipemidic acid, rosoxacin, enoxacin, fleroxacin, iomefloxacin, nadifloxacin, ofloxacin, pefloxacin, rufloxacin, balofloxacin, gatifloxacin, grepafloxacin, levofloxacin, moxifloxacin, pazufloxacin, sparfloxacin, temafloxacin, tosufloxacin, clinafloxacin, garenoxacin, gemifloxacin, sitafloxacin, trovafloxacin, prolifloxacin, and ecinofloxacin.

In some embodiments, coumermycin A₁, one of the aminocoumarins, is used in the reaction mixtures of the present invention. In other embodiments, another aminocoumarin, novobiocin, is used. Clorobiocin is an additional aminocoumarin that may be useful in the cell-free protein synthesis reaction lysate of this invention. Any combination of two or more of quinolones and/or aminocoumarins, such as those named above, can be used in the reaction lysates of this invention.

The inclusion of the DNA gyrase inhibitor in the reaction mixture of the present invention inhibits the growth of either facilitative anaerobic microorganisms or aerobic microorganisms.

### BRIEF DESCRIPTION OF THE DRAWINGS

FIG. 1: Addition of Ciprofloxacin at 1 μg/ml during lysate preparation does not impact GM-CSF yields.

FIG. 2: Cell-free reaction with 1 μg/mL of Ciprofloxacin performs for 12 h at large scale.

FIG. 3: Effects of Norfloxacin and Coumermycin A1 on GM-CSF yields in cell-free reactions.

FIG. 4: Ciprofloxacin at 5 μg/ml and lower concentration does not impact GM-CSF yields in cell-free reactions.

FIG. 5: Novobiocin at 10 μg/ml or higher concentration negatively impacts GM-CSF yields in cell-free reactions.

### DEFINITIONS

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention, which will be limited only by the appended claims.

As used herein the singular forms “a,” “and,” and “the” include plural referents unless the context clearly dictates otherwise. Thus, for example, reference to “a cell” includes a plurality of such cells and reference to “the protein” includes reference to one or more proteins and equivalents thereof known to those skilled in the art, and so forth. All technical and scientific terms used herein have the same meaning as commonly understood to one of ordinary skill in the art to which this invention belongs unless clearly indicated otherwise.

“Aerobic microorganism” refers to a microorganism that can survive and grow in an oxygenated environment.

“Quinolone” refers to a class of synthetic antibacterial drugs. Quinolones inhibit the bacterial DNA gyrase or the topoisomerase IV enzyme, which functions to inhibit DNA replication and transcription.

“Aminocoumarin” refers to a class of antibiotics made up of a 3-Amino-4,7-dihydroxycumarin ring. Aminocoumarins are competitive inhibitors of DNA gyrase, and function by binding to the B subunit of bacterial DNA gyrase. This competitive binding inhibits the ATP-dependent DNA supercoiling catalyzed by DNA gyrase.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” means the segment of DNA involved in producing a polypeptide chain. It may include regions preceding and following the coding region (leader and trailer) as well as intervening sequences (introns) between individual coding segments (exons).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds having a structure that is different from the general chemical structure of an amino acid, but that functions in a manner similar to a naturally occurring amino acid.

There are various known methods in the art that permit the incorporation of an unnatural amino acid derivative or analog into a polypeptide chain in a site-specific manner, see, e.g., WO 02/086075. Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

“DNA gyrase,” or “gyrase” refers to a type II topoisomerase that introduces negative supercoils into DNA by looping the template so as to form a crossing, then cutting one of the double helices and passing the other through it before releasing the break, which changes the linking number by two in each enzymatic step.

“Facilitative anaerobic microorganism” refers to a microorganism that does not require oxygen for growth, but may utilize oxygen if it is present.

“Gyrase inhibitor” refers to any compound, either chemically synthesized or naturally occurring, that inhibits the function of a gyrase.

“In vitro synthesis” or “cell-free synthesis” refers to synthesis of polypeptides or other macromolecules in a reaction mix comprising biological extracts and/or defined reagents. The reaction mix will comprise a template for production of the macromolecule, e.g., DNA, mRNA, etc.; monomers for the macromolecule to be synthesized, e.g., amino acids, nucleotides, etc.; and co-factors, enzymes and other reagents that are necessary for the synthesis, e.g., ribosomes, uncharged tRNAs, tRNAs charged with native or non-native amino acids, polymerases, transcriptional factors, etc.

“Polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

“Polypeptide synthesis reaction lysate” or “synthesis reaction lysate” or “reaction lysate” or “lysate” is any cell derived preparation comprising the components required for the synthesis of polypeptides. The synthesis reaction lysate will contain protein synthesis machinery, wherein such cellular components are capable of expressing a nucleic acid encoding a desired protein where a majority of the biological components are present following lysis of the cells rather than having been reconstituted. A lysate may be further altered such that the lysate is supplemented with additional cellular components, e.g. amino acids, nucleic acids, enzymes, etc. The lysate may also be altered such that additional cellular components are removed following lysis.

An “expression cassette” is a nucleic acid construct, generated recombinantly or synthetically, with a series of specified nucleic acid elements that permit transcription of a particular polynucleotide sequence in a host cell or in a cell-free transcription/translation system. An expression cassette may be part of a plasmid, viral genome, or nucleic acid fragment. Typically, an expression cassette includes a polynucleotide to be transcribed (e.g., a polynucleotide sequence encoding a polypeptide of interest), operably linked to a promoter (e.g., a T7 promoter from the T7 bacteriophage), which means the promoter sequence is connected to the coding sequence in such a manner (e.g., typically upstream from the coding sequence) that the promoter can function to direct the proper transcription of the coding polynucleotide sequence. Optionally, the expression cassette may include additional elements such as a transcription enhancer, a polyadenylation sequence, and a selection marker (e.g., a gene encoding a protein that confers a drug-resistance to the host cell). If desired, an expression cassette may further comprise a gene encoding a reporter gene (e.g., a luciferase or a green fluorescence protein) under the transcriptional control of the promoter sequence upstream from the coding sequence.

### DETAILED DESCRIPTION OF THE INVENTION

### I. Introduction

The present invention provides methods and compositions useful for in vitro polypeptide synthesis reactions. The methods involve the use of DNA gyrase inhibitors to prevent bacterial contamination in lysates and other raw materials used for in vitro production of polypeptides. The compositions include contamination-free cell lysates for in vitro polypeptide synthesis reactions.

The use of bacterial DNA gyrase inhibitors for in vitro polypeptide synthesis reactions provides a highly effective means to control bacterial contamination within a polypeptide synthesis reaction lysate (herein “reaction lysate”) without interfering with protein synthesis. Such properties allow DNA gyrase inhibitors to enhance the output of desired polypeptides resulting from in vitro translation. Gyrase inhibitors enhance the quantity and quality of the protein product by inhibiting the bacterial growth in the reaction and, thus, inhibiting proteolytic activity. The inhibition of bacterial growth and proteolytic activity preserves the homeostasis of the chemical and physical environment in the cell-free reaction.

The present invention may be practiced using either quinolone (e.g., Ciprofloxacin) or aminocoumarin classes of DNA gyrase inhibitors for enhanced in vitro protein production. The DNA gyrase inhibitors of the present invention effectively inhibit the growth of bacteria, including both facilitative anaerobic and aerobic microorganisms, within the reaction lysate.

Although certain DNA gyrase inhibitors have been tested in vitro for their effects on gene expression (Yang et al., Proc. Natl. Acad. Sci. USA 76(7):3304-3308, 1979), the impact of a gyrase inhibitor on gene expression is believed to vary among different promoters depending upon their structures. The present inventors have discovered the T7 promoter as an ideal promoter for use in a cell-free protein synthesis system where a DNA gyrase inhibitor is included for suppressing bacterial growth, because the T7 promoter has shown superior resistance to interference from various DNA gyrase inhibitors.

### II. Reaction Lysate

The present invention is useful for in vitro production of polypeptides. In commercial scale cell-free systems, bacterial contamination within the reaction lysate will reduce or prevent efficient translation of the desired polypeptide. The use of DNA gyrase inhibitors will prevent bacterial contamination, but will not inhibit the translational machinery. The result is enhanced production of the polypeptide desired to be produced by the in vitro reaction.

### A. DNA Gyrase Inhibitors

The present invention uses DNA gyrase inhibitors to suppress or eliminate bacterial contamination during in vitro polypeptide synthesis reactions. DNA gyrase is one of the topoisomerases, a group of enzymes that catalyze the interconversion of topological isomers of DNA (see generally, Kornberg and Baker, DNA Replication, 2d Ed., W. H. Freeman and Co. (1992); Drlica, Molecular Microbiology 6:425 (1992); Drlica and Zhao, Microbiology and Molecular Biology Reviews 61: 377 (1997)). DNA gyrase itself controls DNA supercoiling and relieves topological stress that occurs when the DNA strands of a parental duplex are untwisted during the replication process. DNA gyrase also catalyzes the conversion of relaxed, closed circular duplex DNA to a negatively superhelical form that is more favorable for recombination. The mechanism of the supercoiling reaction involves the wrapping of gyrase around a region of the DNA, double strand breaking in that region, passing a second region of the DNA through the break, and rejoining the broken strands. Such a cleavage mechanism is characteristic of a type II topoisomerase. The supercoiling reaction is driven by the binding of ATP to the DNA gyrase. The ATP is then hydrolyzed during the reaction. This ATP binding and subsequent hydrolysis cause conformational changes in the DNA-bound DNA gyrase that are necessary for its activity. It has also been found that the level of DNA supercoiling (or relaxation) is dependent on the ATP:ADP ratio. In the absence of ATP, DNA gyrase is only capable of relaxing supercoiled DNA.

Bacterial DNA gyrase is a 400 kilodalton protein tetramer consisting of two A (GyrA) and two B subunits (GyrB). Binding and cleavage of the DNA is associated with GyrA, whereas ATP is bound and hydrolyzed by the GyrB protein. GyrB consists of an amino-terminal domain which has the ATPase activity, and a carboxy-terminal domain that interacts with GyrA and DNA. By contrast, eukaryotic type II topoisomerases are homodimers that can relax negative and positive supercoils, but cannot introduce negative supercoils. Ideally, an antibiotic based on the inhibition of bacterial DNA gyrase would be selective for this enzyme and be relatively inactive against the eukaryotic type II topoisomerases.

The two main categories of DNA gyrase inhibitors are the quinolones and aminocoumarins. Quinolones are synthetic analogs of nalidixic acid and inhibit bacterial DNA synthesis by binding to the GyrA subunit of DNA gyrase, which inhibits overall DNA gyrase function. Aminocoumarins bind to the B subunit of DNA gyrase and inhibits DNA supercoiling by blocking its ATPase activity. The binding site for aminocoumarins lies within the N-terminal region of the DNA gyrase B subunit.

In one embodiment, the quinolone Ciprofloxacin is used to inhibit bacterial contamination in cell-free synthesis reactions. Another embodiment utilizes different DNA gyrase inhibitors belonging to the quinolone class, including cinoxacin, flumequine, nalidixic acid, oxolinic acid, piromidic acid, pipemidic acid, rosoxacin, enoxacin, fleroxacin, iomefloxacin, nadifloxacin, norfloxacin, ofloxacin, pefloxacin, rufloxacin, balofloxacin, gatifloxacin, grepafloxacin, levofloxacin, moxifloxacin, pazufloxacin, sparfloxacin, temafloxacin, tosufloxacin, clinafloxacin, garenoxacin, gemifloxacin, sitafloxacin, trovafloxacin, prolifloxacin, and ecinofloxacin. On the other hand, one or more of the aminocoumarins can be used for the purpose of inhibiting bacterial growth. In some embodiments, coumermycin A1 is used in the reaction mixtures of this invention. In other embodiments, another aminocoumarin, novobiocin, is used. Clorobiocin is an additional aminocoumarin that may be useful for practicing this invention. Also, any one or more of the quinolones may be used in combination with one or more the aminocoumarins, including but not limited to those named above, can be used in this invention.

Embodiments of the present invention are useful to control contaminating growth of any bacterial growth. Some embodiments inhibit either facilitative anaerobic and/or aerobic bacteria.

### B. Lysate Preparation

The present invention utilizes a reaction lysate derived from a host cell for in vitro translation of a target protein. Some embodiments of the present invention are methods of in vitro polypeptide synthesis that require the generation of a reaction lysate in which the polypeptide will be produced. Other embodiments provide the reaction lysate as a composition as described herein.

For convenience, the organism used as a source for the lysate may be referred to as the source organism or host cell. Host cells may be bacteria, yeast, mammalian or plant cells, or any other type of cell capable of protein synthesis. A reaction lysate comprises components that are capable of translating messenger ribonucleic acid (mRNA) encoding a desired protein, and optionally comprises components that are capable of transcribing DNA encoding a desired protein. Such components include, for example, DNA-directed RNA polymerase (RNA polymerase), any transcription activators that are required for initiation of transcription of DNA encoding the desired protein, transfer ribonucleic acids (tRNAs), aminoacyl-tRNA synthetases, 70S ribosomes, N¹⁰-formyltetrahydrofolate, formylmethionine-tRNAf^{Met} synthetase, peptidyl transferase, initiation factors such as IF-1, IF-2, and IF-3, elongation factors such as EF-Tu, EF-Ts, and EF-G, release factors such as RF-1, RF-2, and RF-3, and the like.

The DNA gyrase inhibitors of the present invention can be added to the reaction at a number of stages prior to and during the protein synthesis reaction. Gyrase inhibitors can be added to raw lysate, as well as during the process of mixing of all of the components of the cell-free reaction mixture. Gyrase inhibitors can also be added during the actual cell-free synthesis reaction. The final gyrase inhibitor concentration in cell-free protein synthesis reactions is such that the bacterial growth is inhibited and the cell-free reaction and its critical components remain active. The optimum inhibitor concentration is found by titration into the cell-free reaction. Gyrase inhibitor concentrations in cell-free reactions are typically, but not necessarily limited to, the range of 100 ng/mL to 100 μg/mL.

A bacterial lysate derived from any strain of bacteria can be used in the methods of this invention. The bacterial lysate can be obtained as follows. The bacteria of choice are grown up overnight in any of a number of growth media and under growth conditions that are well known in the art and easily optimized by a practitioner for growth of the particular bacteria. For example, a natural environment for synthesis utilizes cell lysates derived from bacterial cells grown in medium containing glucose and phosphate, where the glucose is present at a concentration of at least about 0.25% (weight/volume), more usually at least about 1%; and usually not more than about 4%, more usually not more than about 2%. An example of such media is 2YTPG medium, however one of skill in the art will appreciate that many culture media can be adapted for this purpose, as there are many published media suitable for the growth of bacteria such as E. coli, using both defined and complex sources of nutrients. Cells that have been harvested can be lysed by suspending the cell pellet in a suitable cell suspension buffer, and disrupting the suspended cells by sonication, breaking the suspended cells in a French press, or any other method known in the art useful for efficient cell lysis. The cell lysate is then centrifuged or filtered to remove large DNA fragments.

Rabbit reticulocyte cells provide an example of a mammalian cell type that may be used to generate a lysate. Reticulocyte lysate is prepared following the injection of rabbits with phenylhydrazine, which ensures reliable and consistent reticulocyte production in each lot. The reticulocytes are purified to remove contaminating cells, which could otherwise alter the translational properties of final lysate. The cells can then be lysed by suspending the cell pellet in a suitable cell suspension buffer, and disrupting the suspended cells by sonication, breaking the suspended cells in a French press, or any other method known in the art useful for efficient cell lysis. After the reticulocytes are lysed, the lysate is treated with micrococcal nuclease and CaCl₂ in order to destroy endogenous mRNA and thus reduce background translation. EGTA is further added to chelate the CaCl₂ thereby inactivating the nuclease. Hemin may also be added to the reticulocyte lysate because it is a suppressor of an inhibitor of the initiation factor eIF2α. In the absence of hemin, protein synthesis in reticulocyte lysates ceases after a short period of incubation (Jackson, R. and Hunt, T. 1983 Meth. In Enzymol. 96, 50). Potassium acetate and magnesium acetate are added at a level recommended for the translation of most mRNA species. For further detail on preparing rabbit reticulocyte lysate, one skilled in the art can refer to, e.g., Sambrook et al., Molecular Cloning: A Laboratory Manual, Second Edition (Cold Spring Harbor Laboratory Press, Cold Spring Harbor, N.Y. 1989).

Wheat germ provides a plant cell that may be used as a host for which to generate a lysate that may be used by the methods of the present invention. Generally, wheat germ lysate is prepared by grinding wheat germ in an extraction buffer, followed by centrifugation to remove cell debris. The supernatant is then separated by chromatography from endogenous amino acids and plant pigments that are inhibitory to translation. The lysate is also treated with micrococcal nuclease to destroy endogenous mRNA, to reduce background translation to a minimum. The lysate contains the cellular components necessary for protein synthesis, such as tRNA, rRNA and initiation, elongation, and termination factors. The lysate is further optimized by the addition of an energy generating system consisting of phosphocreatine kinase and phosphocreatine, and magnesium acetate is added at a level recommended for the translation of most mRNA species. For more detail on the preparation of wheat germ lysate, see e.g., Roberts, B. E. and Paterson, B. M. (1973), Proc. Natl. Acad. Sci. U.S.A. Vol. 70, No. 8, pp. 2330-2334), following the modifications described by Anderson, C. W., et al., Meth. Enzymol. (Vol. 101, p. 635; 1983).

Lysates are also commercially available from manufacturers such as Promega Corp., Madison, Wis.; Stratagene, La Jolla, Calif.; Amersham, Arlington Heights, Ill.; and GIBCO, Grand Island, N.Y.

### III. In Vitro Polypeptide Synthesis Reaction

In vitro polypeptide synthesis reactions require the generation of a reaction lysate as described above. It is desired that this reaction lysate be free from bacterial contamination, and it is for this purpose the reaction mixture is supplemented with one or more DNA gyrase inhibitors, although the inhibitor or inhibitors may be added at any stage from the beginning of cell harvest to the extraction step of synthesized protein. Following the synthesis reaction, the resultant polypeptide requires purification. Lastly, the present invention may further be utilized with any in vitro polypeptide synthesis reaction independent of the characteristics of the desired polypeptide, e.g., proteins containing non-native amino acids.

It is anticipated that the invention will find value in reaction systems of between 1 and 10 liters or greater, e.g., 100 liters. In some case, the invention is also practiced in systems of much smaller volumes, for instance in the range of liter to milliliter, or even milliliter to microliter, in various small scale screening applications. The invention will find further value in systems (of either large or small volume) that are allowed to continuously produce protein for 1 to 10, for 1 to 24 hours, or greater, such as 1 to 3 days.

### A. Reaction Conditions

Embodiments of the present invention utilize an in vitro polypeptide synthesis reaction to generate desired proteins. Such reactions utilize reaction lysates generated as described above.

In addition to the DNA gyrase inhibitors, the lysate reaction mixture will comprise monomers for the macromolecule to be synthesized, e.g., amino acids, nucleotides, etc., and such co-factors, enzymes and other reagents that are necessary for the synthesis, e.g., ribosomes, tRNA, polymerases, aminoacyl-tRNA synthetases, transcriptional factors, etc. tRNAs may be aminoacylated in a separate reaction, or directly in the lysate. In addition to the above components such as a cell-free lysate, genetic template, and amino acids, materials specifically required for protein synthesis may be added to the reaction. The materials include salts, folinic acid, cyclic AMP, inhibitors for protein or nucleic acid degrading enzymes, inhibitors or regulators of protein synthesis, adjusters of oxidation/reduction potentials, non-denaturing surfactants, buffer components, spermine, spermidine, putrescine, etc. Metabolic inhibitors to undesirable enzymatic activity may be added to the reaction mixture. Alternatively, enzymes or factors that are responsible for undesirable activity may be removed directly from the extract, or the gene encoding the undesirable enzyme may be inactivated or deleted from the chromosome.

The template for cell-free protein synthesis can be either mRNA or DNA. The template can encode for any particular gene of interest, and may encode a full-length polypeptide or a fragment of any length thereof. A DNA template that comprises the gene of interest will be operably linked to at least one promoter and to one or more other regulatory sequences including without limitation repressors, activators, transcription and translation enhancers, DNA-binding proteins, etc. Nucleic acids to serve as sequencing templates are optionally derived from a natural source or they can be synthetic or recombinant. For example, DNAs can be recombinant DNAs, e.g., plasmids, viruses or the like. Suitable quantities of DNA template for use herein can be produced by amplifying the DNA in well known cloning vectors and hosts, or by polymerase chain reaction (PCR).

Wherein DNA templates are used to drive in vitro protein synthesis, the individual components of the protein synthesis reaction mixture may be mixed together in any convenient order. Optionally, an RNA polymerase is added to the reaction mixture to provide enhanced transcription of the DNA template. RNA polymerases suitable for use herein include any RNA polymerase that functions in the bacteria from which the bacterial extract is derived. In embodiments wherein an RNA template is used to drive in vitro protein synthesis, the components of the reaction mixture can be mixed together in any convenient order, but are preferably mixed in an order wherein the RNA template is added last.

The polypeptide synthesis reaction may utilize a large scale reactor, small scale, or may be multiplexed to perform a plurality of simultaneous syntheses. Continuous reactions will use a feed mechanism to introduce a flow of reagents, and may isolate the end-product as part of the process. Batch systems are also of interest, where additional reagents may be introduced to prolong the period of time for active synthesis. A reactor may be run in any mode such as batch, extended batch, semi-batch, semi-continuous, fed-batch and continuous, and which will be selected in accordance with the application purpose.

The reaction mixture can be incubated at any temperature suitable for the transcription and/or translation reactions. The reaction mixture can be agitated or unagitated during incubation. The use of agitation may enhance the speed and efficiency of protein synthesis by keeping the concentrations of reaction components uniform throughout and avoiding the formation of pockets with low rates of synthesis caused by the depletion of one or more key components. The reaction can be allowed to continue while protein synthesis occurs at an acceptable specific or volumetric rate, or until cessation of protein synthesis, as desired. The reaction can be conveniently stopped by incubating the reaction mixture on ice. The reaction can be maintained as long as desired by continuous feeding of the limiting and non-reusable transcription and translation components.

Various cell-free synthesis reaction systems are well known in the art. See, e.g., Kim, D. M. and Swartz, J. R. Biotechnol. Bioeng. 66:180-8 (1999); Kim, D. M. and Swartz, J. R. Biotechnol. Prog. 16:385-90 (2000); Kim, D. M. and Swartz, J. R. Biotechnol. Bioeng. 74:309-16 (2001); Swartz et al., Methods Mol. Biol. 267:169-82 (2004); Kim, D. M. and Swartz, J. R. Biotechnol. Bioeng. 85:122-29 (2004); Jewett, M. C. and Swartz, J. R., Biotechnol. Bioeng. 86:19-26 (2004); Yin, G. and Swartz, J. R., Biotechnol. Bioeng. 86:188-95 (2004); Jewett, M. C. and Swartz, J. R., Biotechnol. Bioeng. 87:465-72 (2004); Voloshin, A. M. and Swartz, J. R., Biotechnol. Bioeng. 91:516-21 (2005).

While the present invention utilizes DNA gyrase inhibitors during in vitro polypeptide production, the methods and compositions of the present invention can be further used with other techniques useful for enhancing in vitro protein production.

In vitro protein synthesis reactions can exploit the catalytic power of the cellular machinery to further enhance protein production in lysates treated with DNA gyrase inhibitors. Obtaining maximum protein yields in vitro requires adequate substrate supply, e.g., nucleoside triphosphates and amino acids, a homeostatic environment, catalyst stability, and the removal or avoidance of inhibitory byproducts. The optimization of in vitro synthetic reactions benefits from recreating the in vivo state of a rapidly growing organism. In some embodiments of the invention, cell-free synthesis is therefore performed in a reaction where oxidative phosphorylation is activated, i.e., the CYTOMIM™ system. The CYTOMIM™ system is defined by using a reaction condition in the absence of polyethylene glycol and with optimized magnesium concentration.

The CYTOMIM™ system is described in U.S. Pat. No. 7,338,789, herein incorporated by reference. Briefly, the CYTOMIM™ system is defined as a method for in vitro transcription of mRNA and/or translation of polypeptides, the method comprising, synthesizing said mRNA and/or polypeptides in a transcription and/or translation reaction mix substantially free of polyethylene glycol, comprising, an extract from E. coli cells comprising membrane vesicles containing respiratory chain components; components of polypeptide and/or mRNA synthesis machinery; a template for transcription of said mRNA and/or translation of said polypeptide; monomers for synthesis of said mRNA and/or polypeptides; and co-factors, enzymes and other reagents necessary for said transcription and/or translation; magnesium at a concentration of from about 5 mM to about 20 mM; wherein oxidative phosphorylation, which is sensitive to electron transport chain inhibitors, is activated in said reaction mix. The CYTOMIM™ system does not accumulate phosphate, which is believed to inhibit protein synthesis, whereas conventional secondary energy sources result in phosphate accumulation.

In vitro protein synthesis reactions may adjust redox conditions in the reaction mixture to further enhance protein production and folding in lysates treated with DNA gyrase inhibitors. This may include adding a redox buffer to the reaction mix in order to maintain the appropriate oxidizing environment for the formation of proper disulfide bonds. The reaction mixture may further be modified to decrease the activity of endogenous molecules that have reducing activity. Preferably such molecules can be chemically inactivated prior to cell-free protein synthesis by treatment with compounds that irreversibly inactivate free sulfhydryl groups. The presence of endogenous enzymes having reducing activity may be further diminished by the use of extracts prepared from genetically modified cells having inactivation mutations in such enzymes, for example thioredoxin reductase, glutathione reductase, etc. Alternatively, such enzymes can be removed by selective removal from the cell extract during its preparation. Maximizing redox conditions is described in U.S. Pat. Nos. 6,548,276 and 7,041,479, herein incorporated by reference.

In vitro protein synthesis reactions may optimize amino acid concentrations by inhibiting enzymes that act to undesirably metabolize specific amino acids in order to further enhance protein production in lysates supplemented with DNA gyrase inhibitors. Inhibition of enzymes that catalyze the metabolism of amino acids can be achieved by addition of inhibitory compounds to the reaction mix, modification of the reaction mixture to decrease or eliminate the responsible enzyme activities, or a combination of both. A preferred embodiment eliminates arginine decarboxylase. Other such inhibitory compounds to be eliminated from the protein synthesis reaction mixture may include, but are not limited to, tryptophanase, alanine glutamate transaminase, or pyruvate oxidase. Eliminating enzymatic activity in order to optimize amino acid metabolism during cell-free protein synthesis is described in U.S. Pat. No. 6,994,986, herein incorporated by reference.

### B. Purification of Desired Protein

Following the in vitro synthesis reaction, synthesized proteins can be purified from the DNA gyrase inhibitor and other components of the reaction as is standard in the art. Proteins of the invention can be recovered and purified by methods including, but not limited to, ammonium sulfate or ethanol precipitation, acid or base extraction, column chromatography, affinity column chromatography, anion or cation exchange chromatography, phosphocellulose chromatography, hydrophobic interaction chromatography, hydroxylapatite chromatography, lectin chromatography, gel electrophoresis, etc. Newly synthesized proteins containing non-native amino acids must be correctly folded. Proper folding may be accomplished using high performance liquid chromatography (HPLC), affinity chromatography, or other suitable methods where high purity is desired. A variety of purification/protein folding methods are known in the art, e.g., Deutscher, Methods in Enzymology Vol. 182: Guide to Protein Purification (Academic Press, Inc. N.Y. 1990); Bollag et al., Protein Methods, 2nd Edition, (Wiley-Liss, N.Y. 1996). In some cases, one or more DNA gyrase inhibitors, such as those named in this disclosure, can be added to the purification system to minimize or eliminate bacterial contamination.

Following purification, synthesized proteins can possess a conformation different from the desired conformations of the relevant polypeptides. In general, it is occasionally desirable to denature and reduce expressed polypeptides and then to cause the polypeptides to re-fold into the preferred conformation. For example, guanidine, urea, DTT, DTE, and/or a chaperone can be added to a translation product of interest. methods of reducing, denaturing and renaturing proteins are well known to those of skill in the art. See, e.g., Debinski et al., J. Biol. Chem. 268:14065-70 (1993); Buchner et al., Anal. Biochem. 205:263-70 (1992).

The methods of the present invention may provide for modified proteins that have biological activity comparable to the native protein. One may determine the specific activity of a protein in a composition by determining the level of activity in a functional assay, quantitating the amount of protein present in a non-functional assay, e.g. immunostaining, ELISA, quantitation on coomassie or silver stained gel, etc., and determining the ratio of biologically active protein to total protein. Generally, the specific activity as thus defined will be at least about 5% that of the native protein, usually at least about 10% that of the native protein, and may be about 25%, about 50%, about 90% or greater. See, e.g., Sambrook et al., Molecular Cloning: A Laboratory Manual (Cold Spring Harbor Press, Cold Spring Harbor, N.Y. 1989).

Following the in vitro synthesis reaction and subsequent purification, the desired protein produced in a reaction lysate containing a DNA gyrase inhibitor may be optionally used, e.g., as assay components, therapeutic reagents, or as immunogens for antibody production.

### C. Desired Protein Products

The present invention provides methods and compositions for in vitro polypeptide production that can be used to generate any type of protein that one skilled in the art may produce using an in vitro polypeptide synthesis platform.

The synthesized protein may be homologous to, or may be exogenous, meaning that they are heterologous, i.e., foreign, to the cells from which the cell-free lysate is derived, such as a human protein, viral protein, yeast protein, etc. produced in a bacterial cell-free lysate. Modified proteins may include, but are not limited to, molecules such as, e.g., renin, a growth hormone, including human growth hormone; bovine growth hormone; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; clotting factors such as factor VIIIC, factor IX, tissue factor, and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; tumor necrosis factor-alpha and -beta; enkephalinase; RANTES (regulated on activation normally T-cell expressed and secreted); human macrophage inflammatory protein (MIP-1-alpha); a serum albumin such as human serum albumin; mullerian-inhibiting substance; relaxin A-chain; relaxin B-chain; prorelaxin; mouse gonadotropin-associated peptide; a microbial protein, such as beta-lactamase; DNase; inhibin; activin; vascular endothelial growth factor (VEGF); receptors for hormones or growth factors; integrin; protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor such as NGF-(3; platelet-derived growth factor (PDGF); fibroblast growth factor such as aFGF and bFGF; epidermal growth factor (EOF); transforming growth factor (TGF) such as TGF-alpha and TGF-beta, including TGF-(31, TGF-(32, TGF-(33, TGF-(34, or TGF-(35; insulin-like growth factor-I and —II (IGF-I and IGF-II); des(1-3)-IGF-I (brain IGF-I), insulin-like growth factor binding proteins; CD proteins such as CD-3, CD-4, CD-8, and CD-I 9; erythropoietin; osteoinductive factors; immunotoxins; a bone morphogenetic protein (BMP); an interferon such as interferon-alpha, -beta, and -gamma; colony stimulating factors (CSFs), e.g., M-CSF, GM-CSF, and G-CSF; interleukins (ILs), e.g., IL-1 to IL-10; superoxide dismutase; T-cell receptors; surface membrane proteins; decay accelerating factor; viral antigen such as, for example, a portion of the AIDS envelope; transport proteins; homing receptors; addressins; regulatory proteins; antibodies; and fragments of any of the above-listed polypeptides.

The in vitro synthesis of proteins containing non-native amino acids may also be produced using a lysate containing one or more DNA gyrase inhibitors. Non-native amino acids refer to amino acids that are not one of the twenty naturally occurring amino acids that are the building blocks for all proteins that are nonetheless capable of being biologically engineered such that they are incorporated into proteins. Non-native amino acids may include D-peptide enantiomers or any post-translational modifications of one of the twenty naturally occurring amino acids. A wide variety of non-native amino acids can be used in the methods of the invention. The non-native amino acid can be chosen based on desired characteristics of the non-native amino acid, e.g., function of the non-native amino acid, such as modifying protein biological properties such as toxicity, biodistribution, or half life, structural properties, spectroscopic properties, chemical and/or photochemical properties, catalytic properties, ability to react with other molecules (either covalently or noncovalently), or the like. Non-native amino acids that can be used in the methods of the invention may include, but are not limited to, an non-native analogue of a tyrosine amino acid; an non-native analog of a glutamine amino acid; an non-native analog of a phenylalanine amino acid; an non-native analog of a serine amino acid; an non-native analog of a threonine amino acid; an alkyl, aryl, acyl, azido, cyano, halo, hydrazine, hydrazide, hydroxyl, alkenyl, alkynl, ether, thiol, sulfonly, seleno, ester, thioacid, borate, boronate, phospho, phosphono, phosphine, heterocyclic, enone, imine, aldehyde, hydroxylamine, keto, or amino substituted amino acid, or any combination thereof; an amino acid with a photoactivatable cross-linker; a spin-labeled amino acid; a fluorescent amino acid; an amino acid with a novel functional group; an amino acid that covalently or noncovalently interacts with another molecule; a metal binding amino acid; a metal-containing amino acid; a radioactive amino acid; a photocaged and/or photoisomerizable amino acid; a biotin or biotin-analog containing amino acid; a glycosylated or carbohydrate modified amino acid; a keto containing amino acid; amino acids comprising polyethylene glycol or polyether; a heavy atom substituted amino acid; a chemically cleavable or photocleavable amino acid; an amino acid with an elongated side chain; an amino acid containing a toxic group; a sugar substituted amino acid, e.g., a sugar substituted serine or the like; a carbon-linked sugar-containing amino acid, e.g., a sugar substituted serine or the like; a carbon-linked sugar-containing amino acid; a redox-active amino acid; an α-hydroxy containing acid; an amino thio acid containing amino acid; an a, a disubstituted amino acid; a β-amino acid; a cyclic amino acid other than proline, etc.

All publications and patent applications cited in this specification are herein incorporated by reference as if each individual publication or patent application were specifically and individually indicated to be incorporated by reference.

### EXAMPLES

The following examples are provided by way of illustration only and not by way of limitation. Those of skill in the art will readily recognize a variety of non-critical parameters that could be changed or modified to yield essentially the same or similar results.

### Example 1

### Obtaining a Template

The present invention requires the use of a nucleic acid template for the cell-free protein synthesis reaction. The following provides an example of generating a template having codon sequences constructed based on placement of non-native amino acids within the desired polypeptide.

An amino acid sequence of human granulocyte macrophage colony stimulating factor (hGMCSF) was obtained from Research Collaboratory for Structural Bioinformatics (RCSB) protein data bank (PDB). A structural DNA gene encoding hGMCSF protein followed by a 6X-HIS purification tag encoding sequence was synthesized de novo (DNA 2.0, Menlo Park, Calif.). The gene was flanked by the T7 promoter and terminator and was inserted into a plasmid vector containing an E. coli origin of replication and kanamycin resistance gene. The circular plasmid DNA template was prepared by transforming XL1Blue (Stratagene, La Jolla, Calif.) strain of E. coli, growing up the culture at 37° C. overnight on LB media containing 40 μg/mL kanamycin and purifying DNA using a purification kit (Qiagen, Valencia, Calif.).

### Example 2

### Generating an In Vitro Protein Synthesis Lysate

In vitro protein synthesis reactions occurred in lysates generated from a population of cells. The lysate must be generated such that it would be useful for expressing proteins. E. coli A19ΔendAΔtonAΔspeAΔtnaAΔsdaAΔsdaBΔgshAΔgorTrxBHAmet⁺ was used for as the extract source cell strain.

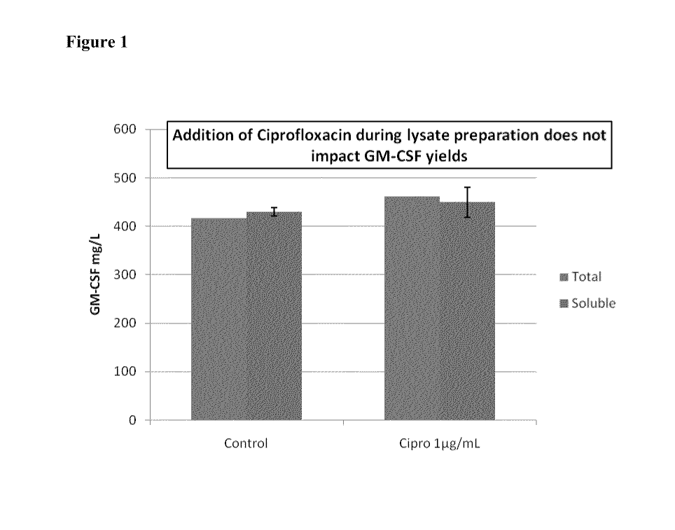

E. coli cells were grown in a 10 L Braun Biostat C fermentor. The cells were grown on 2YPTG media in batch mode with pH control at pH 7.0. The cells were harvested at 3.2 OD (595) at growth rate of >0.7 per hour. Cells were separated from the media by centrifugation at 6000 g, 4° C. for 25 min and the resulting cell paste was stored at −80° C. The cell paste was thawed at 4° C. in S30 buffer (10 mM TRIS-acetate pH 8.2 (Sigma-Aldrich Corp. St. Louis, Mo.), 14 mM magnesium acetate (Sigma-Aldrich), and 60 mM potassium acetate (Sigma-Aldrich)) at a ratio of 1 mL of buffer per 1 g of wet cell paste. Resuspended cells were passed through a high pressure homogenizer (Emulsiflex C-50, Avestin Inc., Ottawa, Ontario, Canada). The pressure drop was set at 20000 psi. The homogenized mixture was then centrifuged at 30000 g, 4° C. for 30 minutes. The procedure was repeated twice and the supernatant was retained both times. The mixture was incubated at 37° C. for 80 min in a rotary shaker. The lysate was then centrifuged at 20000 g, 4° C. for 30 minutes and supernatant was retained. FIG. 1 shows the addition of Ciprofloxacin at 1 μg/ml during lysate preparation did not impact the ability of lysate to produce protein when compared to a lysate without antibiotic.

### Example 3

### In Vitro Protein Synthesis Reaction

Following the generation of a lysate useful for a cell-free protein synthesis reaction as described in Example 2, the desired polypeptide was produced. The cell-free protein synthesis reaction contained the reagents summarized in Table 2.

The extract was pretreated with 50 μM iodoacetamide at 21° C. for 30 min. The plasmid contained the structural gene encoding the target protein and was constructed as explained above. Amino Acids were in an equimolar mixture of all 20 native amino acids. Gyrase inhibitor Ciprofloxacin (Sigma, St. Louis, Mo.) was added at concentration of 1 μg/mL to the cell-free reaction mixture.

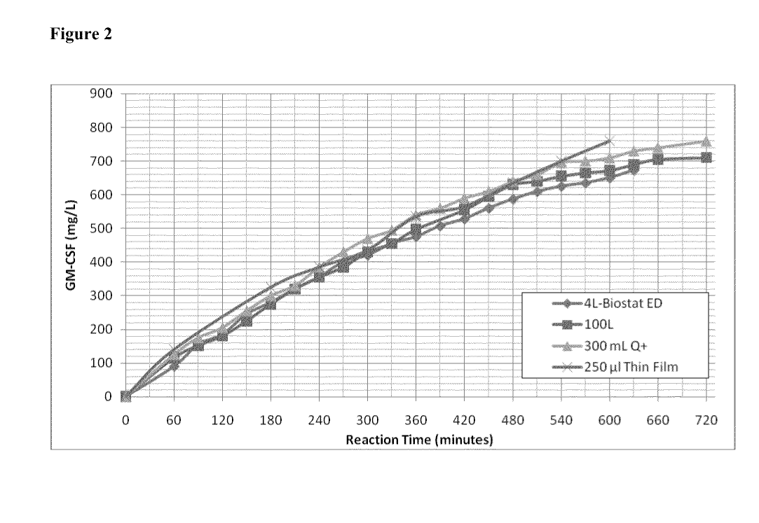

The 250 μL of reaction mixture is spread on the bottom of a petri dish (BD Falcon) and incubated at 30° C. in a sealed humidified incubator for 5 hours. 300 mL reaction is performed in QPlus bioreactor, 4 L in 10 L Braun Biostat C fermentor and 100 L in 200 L fermentor (Sartorius). FIG. 2 shows the GM-CSF yields in cell-free reactions performed for 12 h at four different scales with Ciprofloxacin added to them at 1 μg/ml.

TABLE 1
Summary of Reagents added the Cell-free Protein Expression system

| Reagent | Concentration |
|---|---|
| Magnesium Glutamate | 8 mM |
| Ammonium Glutamate | 10 mM |
| Potassium Glutamate | 130 mM |
| AMP | 1.20 mM |
| GMP | 0.86 mM |
| UMP | 0.86 mM |
| CMP | 0.86 mM |
| Amino Acids | 2 mM |
| Ciprofloxacin | 1 μg/mL |
| Pyruvate | 30 mM |
| NAD | 3.3 mM |
| CoA | 2.7 mM |
| Oxalic Acid | 4 mM |
| Spermidine | 1.5 mM |
| Putrescine | 1 mM |
| T7 RNA polymerase | 0.10 mg/ml |
| Plasmid | 0.0133 mg/ml |
| E. coli DsbC | 75 ug/ml |
| E. coli extract | 6/25 total reaction volume |

### Example 4

### Synthesized Protein Purification

Following the in vitro protein synthesis reaction, the desired polypeptide must be purified from the reaction mixture. To purify the GMCSF-6X-HIS protein out of the completed cell-free reaction mixture, the mix was diluted 10 fold with an appropriate buffer and was loaded onto an appropriately sized Ni-NTA column equilibrated with equilibration buffer (20 mM Tris-HCl, 0.1 M NaCl, 20 mM Imidazole at pH 7.5). The column was washed with 10 column volumes of wash buffer (20 mM Tris-HCl, 0.1 M NaCl, 60 mM Imidazole at pH 7.5) and the bound protein product was eluted off the column with elution buffer (20 mM Tris-HCl, 0.1 M NaCl, 250 mM Imidazole at pH 7.5).

### Example 5

### Inhibition of Bacterial Growth by Norfloxacin and Coumermycin A₁

Cell-free reactions were performed similar to Example 3 but at 60 μL scale in 24-well plates (BD Falcon) to determine the concentration of Norfloxacin and Coumermycin A₁ antibiotics that can inhibit bacterial growth in cell-free reactions without affecting the ability of lysate to synthesize GM-CSF.

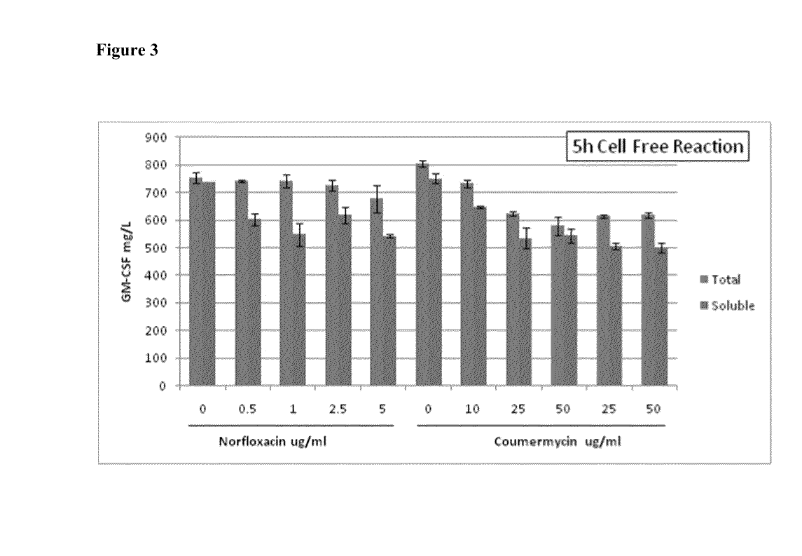

Based on literature inhibitory concentration of Coumermycin A1 (aminocoumarin) for some strains tested was 2-8 μg/ml (Hooper et al., Antimicrobial Agents Chemotherapy, 1982, Vol. 22 (4) p. 662-671) and for Norfloxacin (quinolone) for most gastrointestinal strains tested was between 0.008-1 μg/ml, except for Clostridium difficile, which was 1-128 μg/ml (Shungu et al., Antimicrobial Agents Chemotherapy, 1983, Vol. 23 (1) p. 86-90). Thus 0.5, 1, 2.5, and 5 μg/ml of Norfloxacin (MP Biomedicals) and 10, 25, and 50 μg/ml of Coumermycin A1 (Enzo Life Sciences) were tested in cell-free reactions for GM-CSG synthesis. As shown in FIG. 3, antibiotics Norfloxacin up to 2.5 μg/ml and Coumermycin A1 at 10 ug/ml had very mild impact on GM-CSF yields in cell-free reactions.

20 μl of lysate was inoculated into 2 ml LB medium, which contained 0, 0.1, 0.25, 0.5, and 1 μg/mL of Norfloxacin or 0, 1, 2.5, 5, and 10 μg/ml Coumermycin A1 and was incubated at 30° C. by shaking at 300 rpm. OD595 was assayed after incubation for 20 hours. Table 2 shows that 1 μg/ml Norfloxacin and 10 μg/ml Coumermycin A1 were able to inhibit bacterial growth in lysates used for cell-free reactions.

TABLE 2
Concentration of Norfloxacin and Coumermycin A1 that inhibits
cell-growth from lysate

| Norfloxacin | | Coumermycin A1 | |
|---|---|---|---|
| (μg/ml) | OD595 | (μg/ml) | OD595 |
| 0 | 2.4 | 0 | 2.4 |
| 0.1 | 1.8 | 1 | 1.7 |
| 0.25 | 1.3 | 2.5 | 1.2 |
| 0.5 | 1.2 | 5 | 1.1 |
| 1 | 0.06 | 10 | 0.2 |

### Example 6

### Inhibition of Bacterial Growth by Ciprofloxacin (Cipro) and Novobiocin

20 μl of lysate was inoculated into 2 ml LB medium, which contained Ciprofloxacin or Novobiocin at a certain concentration and was incubated at 37° C. by shaking at 220 rpm for 16 hours. Cell density was assayed by measuring OD595. It was observed that bacterial growth could be inhibited by 1 μg/ml or higher concentration of Ciprofloxacin and 10 μg/ml or higher concentration of Novobiocin as shown in Table 3.

TABLE 3
Cell density in the presence of various concentrations
of Ciprofloxacin and Novobiocin

| Cipro. | | Cipro./HCl* | | Novobiocin | |
|---|---|---|---|---|---|
| (μg/ml) | OD595 | (μg/ml) | OD595 | (μg/ml) | OD595 |
| 0 | 0.56 | 0 | 0.56 | 0 | 0.56 |
| 0.1 | 0.58 | 0.1 | 0.8 | 10 | 0.003 |
| 0.5 | 0.08 | 0.5 | 0.05 | 25 | 0.17 |
| 1 | 0.07 | 1 | 0.02 | 50 | 0.08 |
| 5 | 0.02 | 5 | 0.02 | 100 | 0.1 |
| 10 | 0.04 | 10 | 0.03 | 200 | 0.13 |

*Ciprofloxacin is dissolved in HCl for a higher solubility.

Cell-free reactions were performed similar to Example 5 to determine the concentration of Ciprofloxacin and Novobiocin antibiotics that could inhibit bacterial growth in cell-free reactions without affecting the ability of lysate to synthesize GM-CSF.

Cell-free reactions were performed with 0, 0.5, 1, 2.5, and 5 μg/mL of Ciprofloxacin (Fluka Analytical) and 0, 10, 25, 50, 100, and 200 μg/mL of Novobiocin (Calbiochem) added to the reactions.

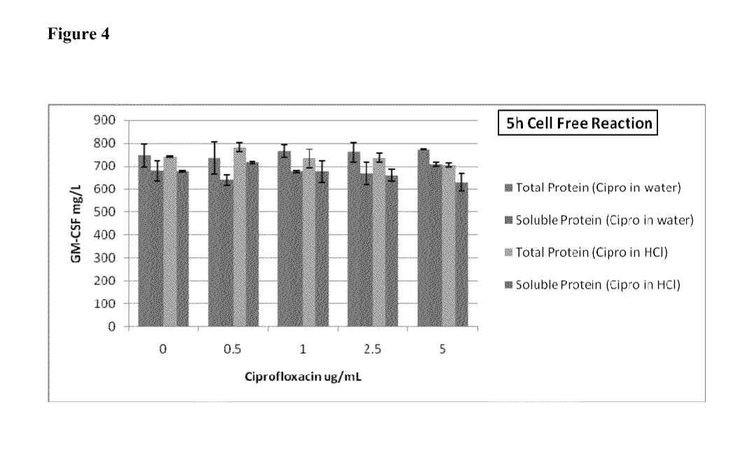

As shown in FIG. 4, Ciprofloxacin at 5 μg/ml and lower concentration did not impact GM-CSF yields in cell-free reactions.

Even though 1 μg/ml of Cipro was sufficient to inhibit bacterial growth, up to 5 μg/ml of Cipro could be tolerated in cell-free reactions.

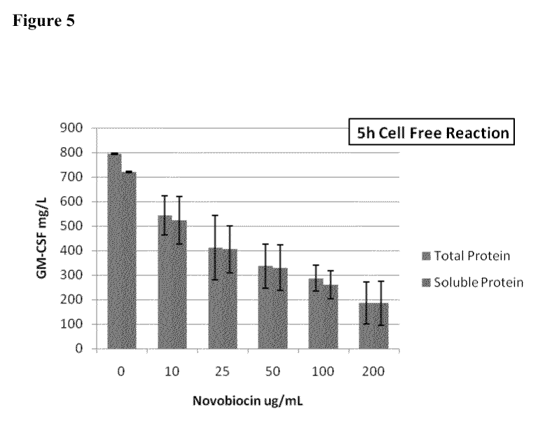

On the other hand, Novobiocin even at 10 μg/ml negatively affected the yields of GM-CSF and the yields were further reduced with increasing Novobiocin concentration as shown in FIG. 5. Literature suggests using 100-300 μg/ml of Novobiocin to inhibit growth of different bacterial strains (Smith and Davis, J Bacteriol. 1967, p 71-79), whereas the minimum inhibitory concentration for ciprofloxacin is about 1 μg/ml (Klein et al., J Vet Diagn Invest, 1996, 8:494-495). Novobiocin at 10 μg/ml or higher concentration negatively impacted protein synthesis in cell-free reactions possibly by affecting glutathione as it was shown that Novobiocin led depletion of hepatic nonprotein sulfhydryl groups (mainly reduced glutathione) in both in vivo and in vitro studies using rats (Lake et al., Toxicol. Applied Pharmacol., 1989 97: 311-323 and Fernyhough et al., Toxicol., 1994, 88:113-125). In the event that no antibiotics other than aminocoumarins are available, it still will be possible to use Novobiocin in a cell-free reaction but the cell-free reagents may require adjustment, for example, addition of GSH, as suggested by the literature to reduce toxicity in hepatic cells as indicated the above-mentioned rat studies.

TABLE 4
Summary of the four antibiotics tested (Examples 5 and 6)

| | | Concentration | |
|---|---|---|---|
| | | that inhibits | Concentration |
| | | growth (μg/ml) | without impact in |
| | | (According to | cell-free reactions |
| Antibiotic | Class | literature) | (μg/ml) |
| Ciprofloxacin | Quinolone | 1 (1) | 5 |
| Norfloxacin | Quinolone | 1 (0.008-1) | 2.5 |
| Novobiocin | Aminocoumarin | 10 (100-300) | — |
| Coumermycin | Aminocoumarin | 10 (2-8) | 10 |
| A1 | | | |

What is claimed is:

1. A method for in vitro synthesis of a polypeptide comprising adding a DNA gyrase inhibitor to a polypeptide synthesis reaction lysate in an amount sufficient to inhibit bacterial growth, wherein the polypeptide is synthesized from an expression cassette comprising a polynucleotide sequence encoding the polypeptide operably linked to a T7 promoter.

2. The method of claim 1, wherein the DNA gyrase inhibitors is a quinolone.

3. The method of claim 2, wherein the quinolone is ciprofloxacin.

4. The method of claim 2, wherein the quinolone is norfloxacin.

5. The method of claim 2, wherein the quinolone is selected from the group consisting of cinoxacin, flumequine, nalidixic acid, oxolinic acid, piromidic acid, pipemidic acid, rosoxacin, enoxacin, fleroxacin, iomefloxacin, nadifloxacin, ofloxacin, pefloxacin, rufloxacin, balofloxacin, gatifloxacin, grepafloxacin, levofloxacin, moxifloxacin, pazufloxacin, sparfloxacin, temafloxacin, tosufloxacin, clinafloxacin, garenoxacin, gemifloxacin, sitafloxacin, trovafloxacin, prolifloxacin, and ecinofloxacin.

6. The method of claim 1, wherein the DNA gyrase inhibitor is an aminocoumarin.

7. The method of claim 6, wherein the aminocoumarin is novobiocin.

8. The method of claim 6, wherein the aminocoumarin is coumermycin.

9. The method of claim 1, wherein the DNA gyrase inhibitor inhibits the growth of facilitative anaerobic microorganisms.

10. The method of claim 1, wherein the DNA gyrase inhibitor inhibits the growth of aerobic microorganisms.

11. An in vitro polypeptide synthesis reaction mixture comprising (1) a DNA gyrase inhibitor in an amount sufficient to inhibit bacterial growth, and (2) an expression cassette comprising a polynucleotide sequence encoding a polypeptide to be synthesized operably linked to a T7 promoter.

12. The reaction mixture of claim 11, wherein the DNA gyrase inhibitor is a quinolone.

13. The reaction mixture of claim 12, wherein the quinolone is ciprofloxacin.

14. The reaction mixture of claim 12, wherein the quinolone is norfloxacin.

15. The reaction mixture of claim 12, wherein the quinolone is selected from the group consisting of cinoxacin, flumequine, nalidixic acid, oxolinic acid, piromidic acid, pipemidic acid, rosoxacin, enoxacin, fleroxacin, iomefloxacin, nadifloxacin, ofloxacin, pefloxacin, rufloxacin, balofloxacin, gatifloxacin, grepafloxacin, levofloxacin, moxifloxacin, pazufloxacin, sparfloxacin, temafloxacin, tosufloxacin, clinafloxacin, garenoxacin, gemifloxacin, sitafloxacin, trovafloxacin, prolifloxacin, and ecinofloxacin.

16. The reaction mixture of claim 11, wherein the DNA gyrase inhibitor is an aminocoumarin.

17. The reaction mixture of claim 16, wherein the aminocoumarin is novobiocin.

18. The reaction mixture of claim 16, wherein the aminocoumarin is coumermycin.

19. The reaction mixture of claim 11, wherein the DNA gyrase inhibitor inhibits the growth of facilitative anaerobic microorganisms.

20. The reaction mixture of claim 11, wherein the DNA gyrase inhibitor inhibits the growth of aerobic microorganisms.
